(19) United States
(12) Patent Application Publication
(10) Pub. No.: US 2003/0165926 A1
(43) Pub. Date: Sep. 4, 2003

(54) Method for identifying and isolating genome fragments with coupling disequilibrium

(76) Inventors: Klaus Olek, Bonn (DE); Jurgen Weber, Neuwied (DE); Thomas Jansen, Monchengladbach (DE); Marco Leuer, Bonn (DE)

Correspondence Address: KRIEGSMAN & KRIEGSMAN, 665 FRANKLIN STREET, FRAMINGHAM, MA, 01702, US

(21) Appl. No.: 10/257,168

(22) PCT Filed: Apr. 8, 2001
(86) PCT No.: PCT/DE01/01488

(30) Foreign Application Priority Data
Apr. 8, 2000 (DE) 100 17 675.5

(51) Int. Cl. C12Q 1/68

(52) U.S. Cl. 435/6

(57) ABSTRACT

The subject of the invention is a method for the identification and isolation of genome fragments with coupling disequilibrium, wherein regions of the genome are isolated, which contain candidate gene regions that are found in coupling disequilibrium with their constricted DNA environment, and these genome regions are obtained from individuals who are not related to one another as well as individuals who are related to one another.

The earlier conducting of the cloning step, in comparison to other methods, makes possible an obtaining of DNA fragments that is independent of quantity and in addition replaces the methylation step conducted in other methods. It was also found that the use of a plant enzyme according to the invention brings about a much higher specificity of the method when compared with those in which the Mut S, H, L complex is used.

The invention concerns a method for identifying and isolating genome fragments with coupling disequilibrium.

Modem animal breeding for several years has made use of molecular biological analysis. Great expectations have been coupled to the strategy of marker-supported breeding. In addition to the clinically and genetically often precisely described monogenic inherited disorders, the target field of these efforts includes the economically highly attractive QTLs. The latter involve so-called multifactorial features, which are simultaneously controlled by several genetic influence factors, but also by external factors. In the broadest sense, these are the performance parameters of animal breeding. The currently most attractive factors are, for example, milk capacity, meat quantity, growth and fertility. In the first 5 years of worldwide systematic application of marker-supported breeding, the results, however, have been somewhat disappointing; the method obviously possesses narrow limits in principle and is very expensive.

Selection strategies of useful-animal breeding, which have been exercised for a long time, have been contrasted for several years to an alternative that is based on molecular biological methods, which is named marker-assisted selection (in English: marker-assisted selection or briefly, MAS). The core concept involves the application of coupling strategy with polymorphic DNA markers. The latter has proven very successful in the discovery of monogenic hereditary diseases in humans. The strategy has been systematically applied worldwide as a first step of the Human Genome Program (HGP). The theoretical principles are the considerations of Botstein et al., 1980. The enormous efforts of the HGP finally supplied the technical and intellectual basis for determining that a marker map could be constructed for the important useful-animal species of cow, pig and sheep. There are now available approximately 2,000 localized polymorphic markers within the bovine genome. There are more than 1,200 in the swine genome. These involve mostly microsatellites. With an assortment of 300 of these markers, which are distributed as uniformly as possible throughout the entire genome, an instrument is available that permits localizing practically any monogenic inherited and phenotypically clearly described feature. However, for this, several hundred affected animals must be typed with this marker set, i.e., all 300 marker-gene loci must be analyzed for the animals. Usually, the site of the causative gene in the genome is found by means of extensive calculations. A typical result of such calculations is that the site of the gene is constricted to 20-25 cM which corresponds to 20-25 megabases. As a limited practically useful result, one also finds that within one family specific alleles of the closest-lying marker loci are inherited together with the interesting phenotype. Another allele of the same marker gene site may be present in another family. This circumstance can be utilized diagnostically within a family, since such an allele in this family permits genetic predictions on the phenotype to be expected. Such information, however, is associated with a considerable uncertainty due to the phenomenon of recombination between two gene sites. This uncertainty can be reduced by seeking markers, which lie closer to the causative gene. Usually, marker gene sites which lie directly at or in the gene itself are finally found with such a search. Therefore, recombinations almost no longer occur between these marker gene sites and the gene [of interest]. If the defect in the gene of interest can be traced back to one ancestor, this defect is thus passed on by means by a single ancestor, and this defect then spreads from this ancestor to a specific population, and thus one will find the marker allele combination observed at and in the gene in the identical form in all of the affected genes of this population. One thus speaks of the fact that the markers are found in coupling disequilibrium with the ancestor gene. Such a marker allele combination is of extraordinary practical importance, since it permits the desired prediction of the phenotype over and beyond family limits. It is a great disadvantage, however, that the path starting from the coupling group of 25 mB up to [actual localization of] the gene is very arduous and under certain circumstances can take several years, since 25 million base pairs must be studied.

This strategy can also be applied to polygenic inherited features, such as e.g., to the so-called QTLs of useful-animal breeding. The situation now, however, is basically more difficult, not only since one is dealing with several causative genes, in which one must find the gene or coupling disequilibrium, but also since the individual genes influence the phenotype to very different degrees and since a strong environmental influence is also superimposed on the influence allotted to genetics. Therefore, the previous result of several necessarily very large and important QTL studies, each one often with several thousand animals, resulted in the identification of several coupling groups, which are for the most part 30-40 cM in size. They have diagnostic value, but also have value to breeding basically only in families. This means an enormous limitation of the MAS. The ultimate objective of all of these molecular biological efforts, namely the isolation of the genes responsible for the QTLs, cannot practically be obtained on this basis.

Since it could be demonstrated experimentally by a number of research groups that, with the coupling strategy, one could localize not only monogenic inherited disease genes analogously to the studies in the human genome and could make this useful for animal breeding, but also that one could localize the genetically complex QTLs in the genome, the large international breeding programs were adapted to the results that were to be expected for molecular QTL searches. The extremely short time span, in comparison to the conventional strategy, almost exclusively considered the use of bulls viewed as tested as the center of breeding considerations (M. Georges, (1998) Mapping genes underlying production traits in livestock, pp. 77-101. In Animal Breeding: Technology for the 21^{st} century. Ed. J Clarke, Harwood Academic Publishers).

The necessarily very large QTL search programs, which are based on high requirements for the number of animals to be investigated, are nearly all conducted according to a program which is named granddaughter design (GDD) (Gelderman, Theor. Appl. Genet., 46, 319-330 (1975); J. L. Weller, Y. Kashy, M. Soller; J. Dairy Sci., 73, 2525-2537 (1990)).

Only the first two generations will be genotyped, while the phenotyping will occur in the third generation.

The extent of such an investigation will be illustrated below on an example from cattle breeding: a typical number of animals to be investigated is thus 10-20 bulls, 50-100 sons of each of these bulls as well as 50-100 daughters per son.

This means that up to 250,000 genotype analyses are necessary.

The successful application of this method was shown for the first time by M. Georges (M. Georges, D. Nielson, M. Makinnon; Genetics, 139, 907-920 (1995)). The study was conducted at Genmark (USA) with 1,518 bulls in 14 Holstein families with 159 markers.

The GDD studies that were in progress in 1998 are listed in Table 1.

TABLE 1

| Country | Organization | Breed | Families | Sons | Marker | Feature |
|---|---|---|---|---|---|---|
| USA | Genmark | Holstein | 14 | 1518 | 159 | P |
| USA | ABS | Holstein | 14 | 1794 | 224 | P, SCC, |
| | | | | | | L |
| USA, Israel | Illinois Univ. Israel Cattle | Holstein | 18 | 1555 | 100 | P |
| USA | DBDR, USDA | Holstein | 7 | 900 | 16 | P, SCC |
| Canada | Univ. Guelph | Holstein | 7 | 450 | | P |
| NL | Holl. Genetics | Holstein | 20 | 715 | 200 | P |
| New Zealand | Livest. Impro | | | | | |
| Belgium | Univ. Liege | | | | | |
| France, | INRA, Unceia | Holstein | 9 | 1155 | 150 | P, SCC |
| Normandy, | | | | | | |
| Montbeliarde | | | | | | |
| Germany | ADR, 4Univ | Holstein | 16 | | | P, SCC |
| Finland | MTT | Ayrshire | 12 | 493 | | P |
| Norway | As Univ | Norweg. | 6 | 300 | 300 | P |
| | | Red | | | | |
| Sweden | Uppsala Univ | Swedish | 13 | 515 | | P |
| | | Red And | | | | |
| | | White | | | | |

P: Production, T: Type, SCC: Somatic cell count, L: Longevity, F: Fertility

Since most studies are conducted by commercial organizations, it may happen that many of the results will not be published. Basically a problem exists for the identified QTLs: most of them cannot be localized more precisely than approximately 30-40 cM in the genome; in principle, no more than approximately 20 cM can be obtained with the present search strategies. The greater the uncertainty of localization is, the smaller the inheritability will be. Therefore, despite enormous efforts, only relatively few QTLs have been detected in a molecularly reliable manner. In the year 1998, 14 QTLs could be reliably demonstrated by molecular biological means, as demonstrated mostly by several publications that confirmed one another.

In the meantime, it has generally been recognized as a dilemma that, due to the low resolution and precision of the strategies currently available for QTL searches, the desired effective MAS method is not definitively in sight. Michel Georges formulates this recognition, which has been expressed in numerous discussions in recent years at the highest scientific level, in his presentation made in August 1998 in Warsaw, as follows: “We are of the opinion that present mapping resolution is inadequate for the effective marker selection (MAS) and definitely insufficient for positional candidate cloning. Therefore, novel approaches have to be developed to refine the QTL map position.”

The situation at the moment is for this reason predominately unsatisfactory, since there is a particularly high interest in processing numerous features with low inheritability with MAS. An additional experimental problem is still also the fact that a coupling group with a feature of low inheritability can be detected with much more difficulty than one with high inheritability, due to multifaceted, heavy-weighing exogenous influences.

Thus, a method which in principle overcomes this obstacle to inaccurate localization of the coupling group would be of particular interest. Also, in a completely different qualitative sense, molecular, modem useful-animal breeding could be modified in this way, since the particularly interesting QTLs could be treated.

One way to solve the problem is by coupling disequilibrium. If markers can be found, which are in coupling disequilibrium with the respective causative genes, then the reliability of prediction is considerably greater due to the lack of recombination and one can successfully proceed beyond family limits into the area of large populations in the above-described sense (H. Bovenhuis, R. J. Spelman, 49th Annual Meeting, European Association for Animal Production, Aug. 24-27 (1998), Warsaw, Poland).

The same type of problem is found in the human genome in the search for causative genes for polygenic inherited disorders. Since defects in several, and often in very many genes, produce the phenotype of the disease, these [defects] are so frequent that they are called common disorders. Although billions have been spent for several years in applying the coupling strategy to the problems of diabetes, cardiac circulatory disorders or inherited obesity, there has still been no comprehensive success for any of these disorders.

N. Risch, K. Marikangas; Science, 273, 1516-1517 (1996) have investigated this circumstance from the viewpoint of the biostatistician and have come to the conclusion that one must investigate enormous numbers of patients and members of their families with dense marker maps, in order to achieve the desired effect. These investigations would be so extensive that the goal would not be achieved even with large financial support. The efforts are to be evaluated, on the other hand, also from financial considerations due to such failures, since the necessary extremely careful cataloging of phenotypic heterogeneity cannot be applied to large numbers of subjects. The authors come to the conclusion in their description of the situation, which is entitled “Future of Genetic Studies of Complex Human Diseases.” Either the technique of genotyping must make a qualitative leap or a strategy must be found that permits directly discovering the causative genes or the corresponding coupling disequilibria.

The method of genomic mismatch scanning (GMS), which has been described several times recently, represents another method. By means of this concept, regions can be localized in a genome, as with the coupling strategy, which contain genes responsible for specific precisely defined genetic features. The search operation must occur in a population, in which the interesting feature, e.g., a genetic disease, can be attributed to a single ancestor. The corresponding causative gene and its environment in the genome in all of the affected [descendents] in the considered population originate from this single ancestor and the gene is identical [in all of the affected population] in all sequence features. In Anglo-Saxon, the professional expression for this has been expressed as “Identity by Descent” (IBD).

Coupling groups, however, and also coupling disequilibria, are isolated with the GMS method. Two different procedures have been described for this. In the first procedure, an interesting phenotype is defined and studied by means by pairs, of which both individuals originate from one family and have a clearly described degree of relationship, whereby the individual pairs may each originate from genetically heterogeneous populations or families.

If the second procedure is followed, the assumption is made that in a large population, the interesting phenotypic property can be traced back to the same ancestor. In this case, pairings are formed, which do not consist of individuals that are related to one another.

In the first procedure, coupling groups are isolated, while in the second procedure, genomic regions are isolated, which represent a coupling disequilibrium between the interesting gene and a microhaplotype comprised of several polymorphic gene loci.

With one exception, GMS protocols were only used previously for monogenic inherited disorders. In order to study the yield and the reliability of the isolation of IBD fragments, a series of microsatellite loci was subjected to the GMS procedure in one experiment. Thus, in principle, operation on polygenic inherited features was simulated. All of the previously published studies, however, terminate with the localization or the confirmation of a localization of the isolated IBDs, thus the GMS products, which was undertaken earlier by other methods,.

The publication of Nelson in the year 1993 represents the basis for all of these GMS studies. Here, for the first time, a successful application of this protocol on the localization of genes in the example of yeast was reported.

The first successful application in humans was published in 1997. F. Mirzayans (F. Mirzayans, A. J. Mears, W. Guo, W. G. Pearce, M. A. Walter; Am. J. Hum. Genet., 61, 439-448 (1997)) identified the chromosomal region, which should harbor the gene site for iridogoniodysgenesis (IGDA). The finding was worked up in parallel with coupling analysis and GMS. The disorder involves a very rare autosomal dominant eye disease. For the coupling analysis, a family of 80 members was investigated, 40 of whom had the disorder. The entire genome was thus investigated with 300 microsatellites (F. Mirzayans, W. G. Pearce, I. M. MacDonald, M. A. Walter; Am. J. Hum. Genet., 57 (3), 539-548 (1995)). Accordingly, the IGDA gene was localized to 6p. In the next round of localization, 29 microsatellite markers of chromosome 6 were used. The gene locus was thus limited further to 6p25.

IBD fragments from two cousins of the 5th degree from the above-mentioned family were isolated with a GMS protocol that was essentially unmodified when compared to that of Nelson. When PCR was conducted with the appropriate primers, it was shown that 5 of the 7 positive PCR signals that were obtained (7 microsatellites) originated from one chromosomal region, which also has a significant coupling by means of conventional coupling analysis. The IBD fragments with positive PCR signal that lay one behind the other and the significant coupling extended over a region of 6.9 cM.

As an internal control of the GMS experiment, the chromosome 12 was investigated in parallel to chromosome 6. Chromosome 12 did not produce a positive signal.

The determining motive for this successful study of Mirzayans et al. was the attempt to establish GMS as a method by means of which the gene locus of a rare disorder can be localized without conducting and evaluating thousands of microsatellite typings.

A determining prerequisite for such a successful experiment on an individual pair of index persons is the selection of the degree of relatedness of the two individuals.

If the two individuals are too closely related, one must take into consideration very large IBDs, in the limited case (see below), 80% of a chromosome or more. IBDs will also be indicated, which have nothing to do with the interesting feature. On the other hand, if the degree of relatedness is too slight, the IBD region can no longer be revealed with the localizing instrument due to its slight size, since the localizer has too small a resolution. The marker set used by Mirzayans et al. possesses a resolution of 10 cM. This shows the enormous importance of the localizing instrument in the GMS protocol.

The limits of the described method will be discussed in the following. When IBDs are isolated, false-negative and false-positive results occur. In general, the false-negative results will not be discussed, but their occurrence is not surprising. According to the original protocol, 5 μg of DNA of each index individual are used. Although Mirzayans et al. increased the quantity that was used to 100 μg, the yield probably will not amount to more than 100 ng within one reaction batch of this size. However, this involves a population of approximately 500-1000 different fragments, so that one cannot be certain that all of these can be amplified.

In addition, depending on the sequence, the reannealing may be incomplete and thus can simulate a mutation-caused mismatch.

There are several causes for false-negative results. It is known that Mut S does not recognize C-C mismatches, and it is known that Mut S requires a methylated GATC motif in its neighborhood in order to recognize a mismatch. Of course, the latter is not always indicated. Likewise, the function of Mut L,H has not been fully clarified.

As a result of this complex of theoretical and experimental studies, which finally lead to the study of Mirzayans, it can be established that a monogenic, hereditary feature can be localized in a single GMS experiment in the human genome. The size of the detected coupling group is found to be 6.9 cM. The marker map has a resolution of 10 cM and an index family is available, which allows the analysis of cousins of the 5th degree.

However, it is a disadvantage that the method shows a considerable extent of false-positive and false-negative signals, which are obviously unavoidable in the case of the pre-prepared enzyme packaging in the available GMS protocol and the proposed enrichment steps. The application of the previously described operating instructions to multifactorial or to polygenic hereditary features thus appears less promising.

The second genetic targeted direction pursued with GMS is the experiment for directly isolating regions that show coupling disequilibrium. An example of this approach is the study by Vivian G. Cheung (1998). Here, the gene causing the disorder of autosomal-recessive hereditary congenital hyperinsulinism is localized. This disorder occurs at a relatively high frequency in Ashkenazi Jews. For this reason, in this population, this may proceed from a founder effect. The gene codes for the sulfonyl urea receptor. It has been localized to chromosome 11p15.1 by means of conventional methods.

In all, 8 affected persons from 4 families were investigated by two different approaches. In the first approach, pairs were formed from siblings of one family, while in the second approach, the formation of pairs was made between affected individuals of different families. The localization of the IBDs was conducted on a glass chip, which contained inter-Alu bonds of YAC and PAC banks of chromosome 11, ordered according to chromosomal localization.

In the case of the siblings, an IBD segment was obtained, which contained approximately 80% of chromosome 11, whereby the theoretical expectation amounts to 75%. The resolution of the bank amounted to 2 cM outside the already previously known gene site of the SUR1 gene, while in the neighborhood of 11p15.1, it was approximately 0.25 cM. The hybridization data of the unrelated persons were shown from 9 different pairings. The signals extended over approximately 2.5 cM with different extents for the individual pairings. In contrast, 8 pairings have a signal for one clone, while in each of the two adjacent clones, signals of 7 pairs are found for each. From this, a localization precision of 500 bp is derived in the most favorable statistical case. In the case of 4 pairings, several positive signals are found completely outside the SUR1 region.

The most important result in connection with the present project is the knowledge that the mapping of gene loci is possible with GMS by means of coupling disequilibrium. V. Cheung et al. (V. Cheung, J. P. Gregg, K. J. Gogolin-Ewns, J. Bandong, C. A. Stanley, L. Baker, M. J. Higgins, N. J. Nowak, T. B. Shows, W. J. Ewens, S. F. Nelson, R. S. Spielman; Nature Genetics, 18, 3, 225-230 (1998b)) found that without the previously known localization of the gene, several pairings would have had to be further investigated in order to obtain statistically fully secure results.

The determination of the coupling disequilibrium is extremely important. The QTLs previously identified by means of the coupling strategy are frequently not usable, since the marker loci coupled with the interesting features only show coupling but no association. This means that they can be used only for an indirect gene analysis. Of course, it follows from this that they are not suitable for comprehensive use. The time prior to advancing to the associated haplotypes, as one knows from the corresponding problems in human genetics, can be rather long. It is to be expected that it will be impossible in many cases, if one also uses the previously known strategies (Risch et al., 1996).

In this study by V. Cheung, it is particularly clear that rapid and accurate localization plays a determining role and that at the moment the most suitable approach for this is chip hybridization of a good genomic bank with high resolution.

Unfortunately, the method described here can also leave a basic problem unsolved, namely, the problem of the available DNA quantity. All theoretical and practical considerations of the yield at the end of a GMS procedure do not permit a very high optimism; one is always at the limit of detectability of the amplification strategies that are presently available, as will be demonstrated below with a few numbers.

There is no doubt in the international scientific community that GMS or a similar genome scanning method that is satisfactory in all respects will supercede the present coupling strategy for clarifying complex genetic features. In her study applied to a model, V. Cheung (V. Cheung; Genomics, 47, 1-6 (1998a)) determined a number of parameters, which would characterize the result of the application of a GMS procedure to polygenic features.

In this study, a total of 25 separate GMS experiments were conducted on 14 different pairs of grandparents and grandchildren from CEPH families. The individual pairs were investigated with up to 33 different microsatellite loci. The results have been reproduced in repeated experiments.

The most essential parameter is the enrichment factor of the IBD allele. Each gene locus has an allele, which can be inherited either only from the paternal grandfather or from the paternal grandmother. This allele is the respective IBD fragment or IBD allele. A quantitative measurement (area under the peak of the fluorescent signal of the microsatellite locus in measurement with an automatic sequencing apparatus) is performed at two positions for the GMS experiment; it is conducted, first of all, on the total population of heterohybrids after digestion of the homohybrids with restriction enzymes and Exo III and, secondly, on the remaining heterohybrids after separation of the heterohybrid fraction containing erroneous pairings.

Then the sought-after enrichment factor is defined as follows: the ratio of the IBD allele to the non-IBD allele in the total population of heterohybrids forms the denominator of this ratio value; the corresponding ratio in the completely matching GMS fraction forms the numerator.

Three groups of enrichment capacities are distinguished: i.e., greater than 10, 2-10 and less than 2. For all of the microsatellites used and all of the genotypings (122) that are considered, the result is 29% high enrichment, 45% intermediate enrichment, and 26% low enrichment. In contrast, if the observation is related to the individual gene loci that are used (33), then invariably high enrichment is recognized in each experiment in the case of 7 markers (21%), intermediate enrichment with 4 markers (12%) and low enrichment with 7 markers (21%). If 9 markers are used, a variable enrichment, i.e., low to high, is obtained in the course of repeated experiments. Almost no enrichment is obtained with 6 markers.

Obviously, the total procedure is reproducible to a great extent. The enrichment and thus the success of the GMS procedure is dependent on sequence and thus on the gene site, since approximately 18% of the marker loci that are used do not show enrichment. Of course, a generally valid evaluation of efficiency of the method can still not be derived from this, since Pst I fragments were investigated exclusively.

The selection of the restriction cleavage represents a problem in optimization, which is dependent on at least two influence factors. Each DNA molecule which will be removed in the course of the GMS must bear at last one GATC motif. The smaller the fragments are, the higher the probability is that no GATC is present. Vice versa, in FPERT hydrization, complementary DNA strands are harder to find, if many repetitive fragments are contained next to one another. The probability for this increases with the length of the fragment. Fragments greater than 20 kb are obviously decomposed during the reannealing.

Generally, the yield of various preparation steps is not satisfactory with the present protocol in comparison to the original yeast experiment.

This is not surprising, since the yeast genome is 240 times less complex than the human genome, as it contains far fewer repetitive sequences and there are far more natural sequence polymorphisms than the human genome.

This leads to the fact that the FPERT stage is less efficient [in humans than in yeast]. While in one GMS experiment in yeast, approximately 50% of the DNA utilized is recovered after the FPERT as double-stranded molecules (each time approximately 50% heterohybrids and homohybrids), only 10% is recovered in humans. In a typical experiment, this means that approximately 1 μg of DNA is obtained. After digestion of the homohybrids, approximately 500 ng remain. The IBD fragments are separated therefrom and this may involve correspondingly only a few nanograms, and this amount is smaller, the sharper the GMS selection. Also, the proposed PCR amplification that was actually conducted in one case via inter-Alu motifs is found in the problematical limiting range of 1 ng per fragment (Nelson et al., 1989).

The method used up to the present time (GMS) for isolating IBD (identity by descent) regions of the hereditary material thus has a number of difficulties in principle, which particularly interfere with its application to complex genetic features and these are listed once more below:

The methylation of the DNA of one of the hybridization partners, which is necessary for the physical separation of the homohybrids, can be controlled only with difficulty, since essential portions of each genome are methylated. This can lead to considerable losses in yield in restriction digestion of unmethylated DNA.

It is not clear as to what extent the restriction digestion of the methylated fraction is complete.

The Mut S, H, L complex requires a methylation in the neighborhood of a GATC sequence motif for its reaction of recognition and cleavage.

Mut S does not recognize all possible mismatch combinations.

The enzyme complex requires the neighborhood of a GATC motif.

Single-stranded DNA must be isolated with BNCD at two sites for the described GMS method, in which the purification steps are associated with large losses.

The small yield of the total procedure represents the greatest limitation.

The object of the present invention is thus to present a method, which overcomes the disadvantages of the prior art.

The object of the invention is solved by offering a method that serves for the isolation of IBD fragments for polygenic inherited features. The method has improvements in comparison to all of the previously described protocols and can contribute, among other things, to the solution of the above-described problems in animal breeding.

The method of the invention is based on a concept similar to that of GMS.

An example of embodiment of the method according to the invention will be given below.

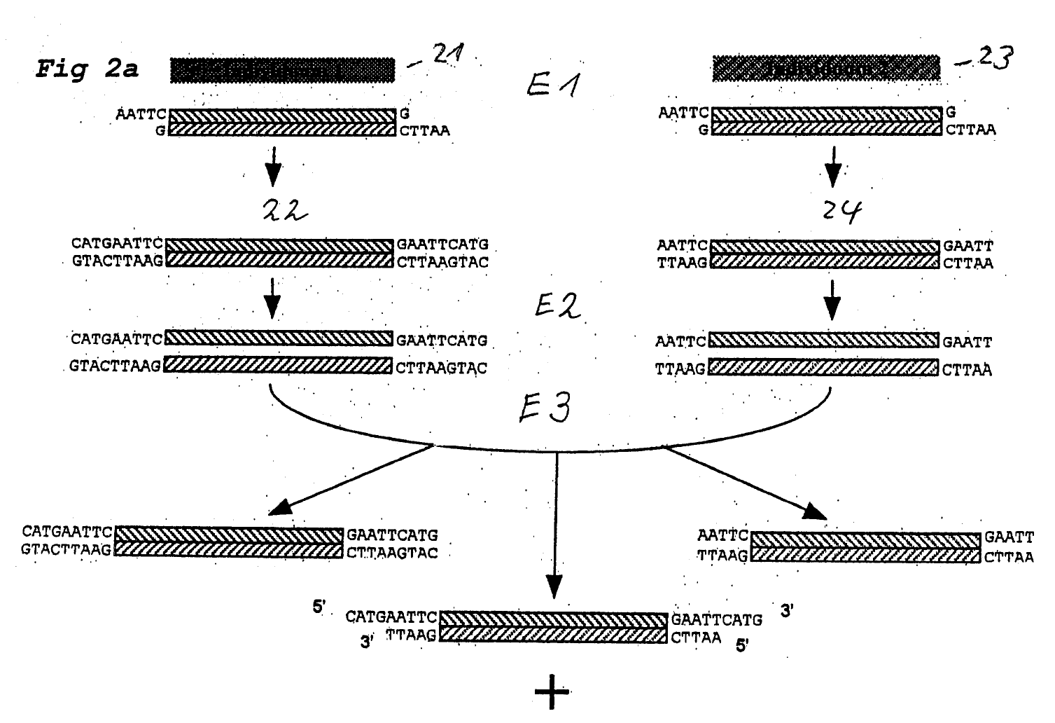

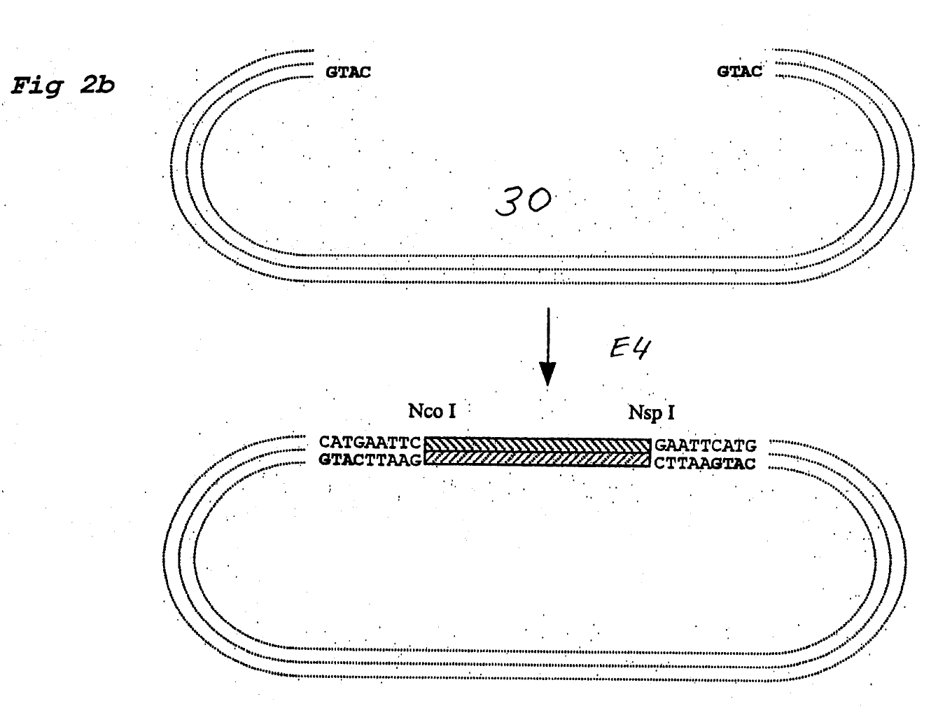

The DNA of two index individuals is extracted and purified. Both DNA fractions are digested with the same restriction enzyme. In the case explained below, for example, in FIG. 2, this involves the restriction enzyme Eco RI. The restricted DNA of individual 1 will be provided with a linker, which produces fragment ends on both sides without an overhang (see FIG. 2). The restricted DNA of individual 2 will also be provided with a linker, which [also] produces fragment ends on both sides without an overhang (see FIG. 2).

The linkers are configured for both individuals in such a way that a self-ligation leading to ring formation is not possible for heterohybrids from the DNA of the two individuals, but in such a way that they allow ring formation with appropriately cut vectors.

The DNA of both individuals is now denatured and the denatured DNA of both individuals is mixed. The mixture is subjected to the so-called FPERT reaction for the reformation of double-stranded DNA. At the end of this reaction, the reaction mixture is comprised of three populations of molecules, namely those from the re-formed double-stranded DNA molecules of individual 1 (homohybrids), the double-stranded DNA molecules of individual 2 (homohybrids) and the double-stranded DNA molecules which are comprised of a DNA strand coming from individual 1 and a DNA strand originating from individual 2 (heterohybrids).

A suitable plasmid vector is subjected to a double digestion, e.g., with the restriction enzymes Nco I and Nsp I. Thus, GTAC ends that stand over the same strand in opposite orientation are formed (see FIG. 2). The mixture of the newly formed double-stranded molecules is combined with the cleaved vector DNA. In this way, only the heterohybrids can form a ring closure with the vector molecules. Then the DNA fragments are coupled covalently with a ligase reaction.

Three populations of molecules are thus formed after this reaction. These include the homohybrids of the two individuals 1 and 2 and the heterohybrids, which are comprised of one strand of individual 1 and the complementary strand of individual 2.

The heterohybrids in turn are comprised of very large groups of molecules which have individual erroneous pairings, and essentially smaller groups of molecules which do not have erroneous pairings. The latter group contains the interesting fragments, namely the IBD regions.

By means of a CEL I reaction, all erroneous base pairings are recognized and are cut out on one strand. The cut-open unpaired regions of the erroneous pairings are then decomposed by means of an exonuclease III step. The total batch is ready for transformation of suitable bacteria without further preliminary purification, and the transformed bacteria are plated out in the appropriate selection medium.

The DNA of the individual plasmid preparations each represents a fragment that is “identical by descent ” (IBD).

The DNA which is obtained is localized on a DNA chip.

A relatively simple application is the identification and the isolation of the gene for the hereditary anal deformity in the domestic pig. Several IBD fragments are obtained with the above-described method according to the invention. These fragments can be localized with the DNA chip. Several gene sites thus result with high probability. These are examined for their information content in affected and unaffected animals. The informative sites can be utilized for diagnostic and ongoing scientific objectives.

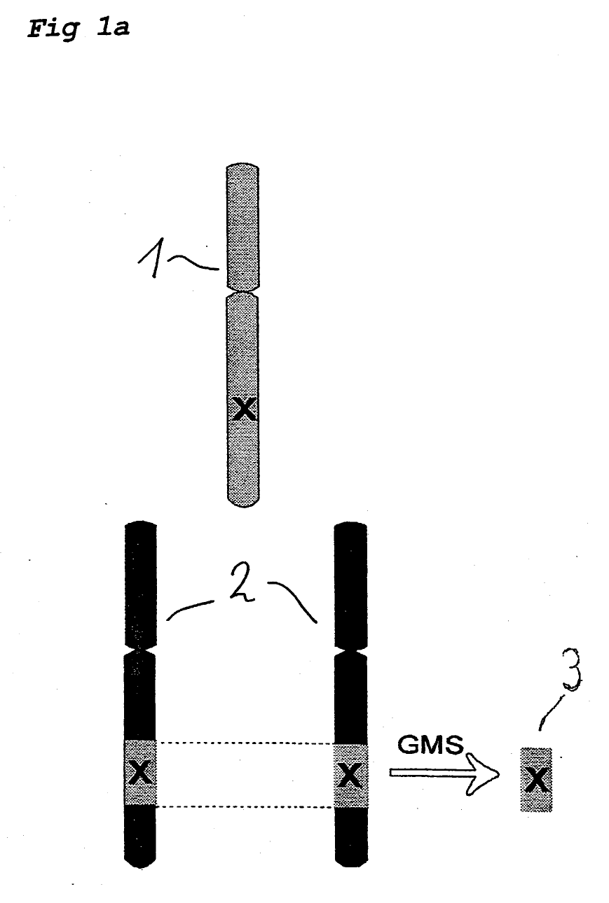

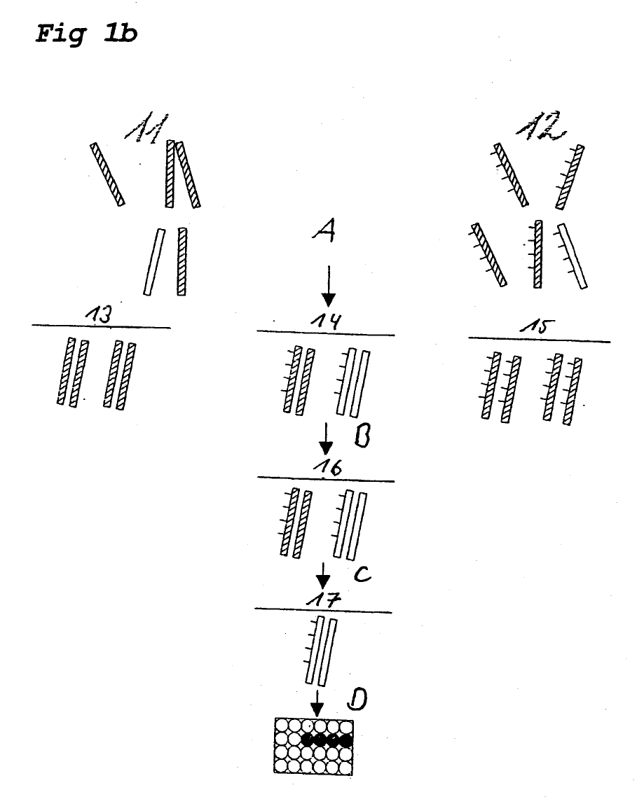

FIGS. 1a and 1b show schematically the steps of the genomic mismatch scanning method.

FIGS. 2a and 2b represent the method according to the invention.

In FIG. 1a, 1 represents the chromosome of an ancestor with several intermediate generations, on which a mutation (X) that is associated with a disorder has appeared. The number 2 indicates the chromosomes of the contemporary individuals with the same disorder, who are not related. 3 represents the region which is “identical by descent” (IBD) and includes the locus for the disorder. In FIG. 1b, 11 represents Pst1 fragments of the first individual and 12 represents the methylated Pst1 fragments of the second individual. 13 then indicates the unmethylated homohybrids, 14 the hemimethylated homohybrids and 15 indicates the methylated homohybrids. Finally, 16 indicates the hemimethylated heterohybrids and 17 the IBD fragments. The reaction steps A, B, C and D are conducted, wherein A is a denaturation and a reannealing; in B, the homohybrids are removed by means of methylation-sensitive restriction enzyme/Exo III digestion; C involves depletion of the heterohybrids containing mismatches with E. coli mismatch repair proteins/Exo III digestion; and D maps the genome location of the GMS-selected IBD fragments on a microarray.

The method according to the invention is schematically shown in FIGS. 2a and 2b. 21 indicates the first individual, while 23 represents the second individual; 22 indicates the first individual plus linker, and 24 indicates the second individual plus linker. 30 designates the vector. The method steps are denoted by E1 to E4 and have the following meanings: E1 is the Eco RI digestion, E2 is naturation, E3 is the mixing of the first and the second individuals with renaturation (FPERT) and E4 designates the ring closure.

It was thus found surprisingly that the conduction of an early cloning step in the method according to the invention makes possible the obtaining of DNA fragments that are independent of quantity and further replaces the methylation step conducted in other methods. It was also found that the use of a plant enzyme according to the invention causes a far higher specificity of the method when compared to those methods in which the Mut S, H, L complex is used.

The object of the invention is thus solved by a method for identifying and isolating genome fragments with coupling disequilibrium, wherein regions of the genome, which contain candidate gene regions that are found in coupling disequilibrium with their constricted DNA surroundings are isolated in individuals who are not related to one another as well as in individuals who are related to one another.

It is preferred that candidate genes that control complex genetic, thus polygenic inherited features, are isolated.

In particular, it is preferred that the DNA samples cut with restriction enzymes from two index individuals are each provided with different linkers, which produce ends without overhang on the two ends of the restriction fragment, but which form overhangs on heterohybrids comprised of fragment ends of the DNA strands that are complementary to one another, each belonging to individual 1 or 2, and these overhangs are found on the same strand and their sequence is 5′ CATG 3′ at the 5′ end and 5′ CATG 3′ at the 3′ end; a mixture is then obtained by denaturing the fragments, which produces single-stranded DNA fragments of the two individuals, and buffer conditions are adjusted in the next step, which permit the renaturing of double-stranded DNA molecules (FPERT reaction); three populations of molecules are obtained, the first of which represents the reproduced double-stranded molecules (homohybrids) of individual 1, the second represents the reproduced double-stranded molecules (homohybrids) of individual 2, and the third represents the population of the double-stranded molecules (heterohybrids) that are complementary to one another, each belonging to one or the other individual; buffer conditions are then adjusted, which permit a ring closure of the molecules that form; in the case of Eco RI digestion, a suitable cloning vector is subjected to a double digestion with the restriction enzymes Nco I and Nsp I; the thus-treated vector DNA is pre-mixed with the reaction mixture from the previous step; a linker with such a specific construction that only the heterohybrids will complete the ring closure with the vector molecules is used; the DNA fragments that are coupled with one another to form a ring are covalently bonded by means of a ligase reaction; ring-form molecules are obtained, which are comprised of two populations, namely those with one or more erroneous base pairings, and those without any erroneous pairings that contain the IBD (identity by descent) regions; the reaction mixture is now combined under suitable buffer conditions with the enzyme CEL I, whereby CEL I recognizes each erroneous pairing and cuts one of the two DNA strands at the position of the erroneous pairing and then the Exo III nuclease is added to this reaction mixture after adjusting suitable buffer conditions, and in this way the rings that are made partially single-stranded by the CEL I digestion are decomposed.

It is also advantageous that this reaction mixture is used for the transformation of suitable bacteria.

It is preferred according to the invention that the transformed bacteria are cultured after selection and isolation and DNA fragments obtained by plasmid preparation and which contain the IBD regions are isolated and localized in the genome.

It is also advantageous that when an enzyme other than Eco RI is used, overhangs of another sequence are obtained, but which have the same orientation.

1. A method for the identification and isolation of genome fragments with coupling disequilibrium, whereby regions of the genome which contain candidate gene regions that are found in coupling disequilibrium with their constricted DNA surroundings are isolated in individuals who are not related to one another as well as in individuals who are related to one another.

2. The method according to claim 1, further characterized in that candidate genes are isolated, which control complex genetic, thus polygenic inherited features.

3. The method according to claim 1, wherein:
a) the DNA samples of two index individuals that are cut with restriction enzymes are each provided with different linkers, which produce ends without overhang on both ends of the restriction fragments, but which form overhangs that are found on the same strand and whose sequence is 5′ CATG 3′ at the 5′ end and 5′ CATG 3′ at the 3′ end;
for heterohybrids comprised of the fragment ends of the DNA strands that are complementary to one another and each of which belongs to individual 1 or 2.
b) by denaturing the fragments, a mixture is obtained, which contains single-stranded DNA fragments of both individuals, and, in the next step, buffer conditions are adjusted, which permit the renaturation of double-stranded DNA molecules (FPERT reaction);
c) three population of molecules are obtained, the first of which represents the reproduced double-stranded molecules (homohybrids) of individual 1, the second represents the reproduced double-stranded molecules (homohybrids) of individual 2 and the third represents the population of the double-stranded molecules (heterohybrids) that are complementary to one another and each of which belongs to one or the other individual;
d) the buffer conditions are adjusted, which permit a ring closure of the molecules that form;
e) in the case of the Eco RI digestion, a suitable cloning vector is subjected to a double digestion with the restriction enzymes Nco I and Nsp I;
f) in addition, the thus-treated vector DNA is mixed with the reaction mixture from step e);
g) a linker with such a specific construction that only the heterohybrids complete the ring closure with the vector molecules is used;
h) the ring-form DNA fragments that are coupled together are covalently bonded by means of a ligase reaction;
i) ring-shaped molecules are obtained, which are comprised of two populations, namely one with one or more erroneous base pairings, and the other without any erroneous pairing that contains the IBD (identity by descent) regions.
j) the reaction mixture is now reacted under suitable buffer conditions with the enzyme CEL I, whereby CEL I recognizes each erroneous pairing and cuts one of the two DNA strands at the position of the erroneous pairing, and
k) the nuclease Exo III is added to this reaction mixture after adjusting suitable buffer conditions, and in this way, the rings that have been partially made single-stranded by the CEL I digestion are decomposed.

4. The method according to claim 3, further characterized in that this reaction mixture is used for the transformation of suitable bacteria.

5. The method according to claim 4, further characterized in that the transformed bacteria are cultured after selection and isolation and that the DNA fragments obtained by plasmid preparations, which contain the IBD regions, are isolated and localized in the genome.

6. The method according to claim 3, further characterized in that overhangs of another sequence are obtained, but which have the same orientation, if an enzyme other than Eco RI is used.
